(19) United States
(12) Patent Application Publication
(10) Pub. No.: US 2026/0335520 A1
(43) Pub. Date: Oct. 29, 2026

(54) EXTRACORPOREAL GAS EXCHANGE SYSTEMS FOR USE WITH PRETERM INFANTS

(71) Applicant: University of Pittsburgh - Of the Commonwealth System of Higher Education, Pittsburgh, PA (US)

(72) Inventors: Katelin Samski, Pittsburgh, PA (US); William J. Federspiel, Tampa, FL (US); Brian Joseph Frankowski, Imperial, PA (US)

(21) Appl. No.: 19/111,517

(22) PCT Filed: Sep. 14, 2023
(86) PCT No.: PCT/US2023/074186

§ 371 Date: Mar. 13, 2025

Related U.S. Application Data
(60) Provisional application No. 63/406,532, filed on Sep. 14, 2022.

(51) Int. Cl. A61M 1/16; A61M 1/36

(52) U.S. Cl. CPC A61M 1/1625; A61M 1/3659; A61M 2202/0007; A61M 2202/0014; A61M 2202/0021; A61M 2202/0208; A61M 2202/0225; A61M 2202/0413; A61M 2205/3334; A61M 2210/12; A61M 2240/00

(57) ABSTRACT

A device to achieve gas exchange when used in connection with premature infants includes a housing and a fiber bundle positioned within a fiber bundle compartment of the housing. Hollow gas permeable fibers of the fiber bundle are adapted to permit diffusion of gas between blood and an interior of the hollow gas permeable fibers. A maximum total resistance of the device is the resistance of a native placenta (that is, for the age of the premature infant) minus the resistance of the flow path connecting the device to the circulatory system of the premature infant.

### CROSS-REFERENCE TO RELATED APPLICATIONS

This application claims benefit of U.S. Provisional Patent Application Ser. No. 63/406,532, filed Sep. 14, 2022, the disclosure of which is incorporated herein by reference.

### GOVERNMENTAL INTEREST

This invention was made with government support under grant number HL076124 awarded by the National Institutes of Health. The government has certain rights in the invention.

### BACKGROUND

The following information is provided to assist the reader in understanding technologies disclosed below and the environment in which such technologies may typically be used. The terms used herein are not intended to be limited to any particular narrow interpretation unless clearly stated otherwise in this document. References set forth herein may facilitate understanding of the technologies or the background thereof. The disclosure of all references cited herein are incorporated by reference.

Preterm infants born before 24 weeks gestational age (GA) have a 95-96% mortality rate and a 98-100% morbidity rate. An infant's chance of survival is positively correlated to its GA and weight at birth. At 25 weeks GA fetuses have a 50% survival chance without medical care. This mortality milestone is known as fetal viability. If extremely premature infants (EPIs) are bridged to viability, the standard of care is to administer exogenous surfactants, antenatal steroids, and respiratory support via ventilation. However, the use of ventilators results in chronic lung disease in all EPIS.

Extracorporeal gas exchange therapy would obviate the need for ventilator support and diminish the incidence of the most prevalent EPI morbidity. Major limitations with the gas exchange devices currently being used in research are priming volumes greater than normal placental volume, device resistances that vary greatly from normal placental physiology, inefficient gas exchange, and lackluster biocompatibility. Others that have successfully completed animal trials using artificial placentas have utilized devices intended for neonates of advanced gestational age.

It is thus desirable to develop extracorporeal gas exchange devices suitable for use with preterm or premature infants, and particularly with extremely premature infants.

### SUMMARY

In one aspect, a device for use in connection with a premature infant to achieve gas exchange includes a housing, and a fiber bundle positioned within a fiber bundle compartment within the housing. The fiber bundle includes a plurality of hollow gas permeable fibers. The plurality of hollow gas permeable fibers is adapted to permit diffusion of gas between blood and an interior of the plurality of hollow gas permeable fibers. The plurality of hollow gas permeable fibers is positioned such that blood flows around the plurality of hollow gas permeable fibers when flowing through the fiber bundle compartment. The housing further includes a gas inlet in fluid connection with the housing and in fluid connection with inlets of the plurality of hollow gas permeable fibers, a gas outlet in fluid connection with the housing and in fluid connection with outlets of the plurality of hollow gas permeable fibers, a blood outlet in fluid connection with the housing and in fluid connection with a first end of the fiber bundle and, a blood inlet in fluid connection with the housing and in fluid connection with a second end of the fiber bundle. The blood outlet is configured to be placed in fluid connection with the circulatory system of the premature infant (for example, via the umbilical vein via cannulation). The blood inlet is configured to be placed in fluid connection with the circulatory system of the premature infant (for example, via at least one of the umbilical arteries via cannulation). In a number of embodiments, the device is configured so that flow of blood through the device may be driven by the heart of the premature infant. No extracorporeal pump is required to achieve flow of blood through the device in a number of embodiments. However, a supplemental, extracorporeal pump can be provided in some embodiments. A maximum total resistance of the device may be the resistance of a native placenta (that is, for the age of the premature infant) minus the resistance of the flow path connecting the device to the circulatory system of the premature infant.

In a number of embodiments, the maximum total resistance of the device is no greater than 100 (mmHg)(min)(L)⁻¹ minus the resistance of the flow path connecting the device to the circulatory system of the premature infant. In a number of embodiments, the maximum total resistance of the device is no greater than 71 (mmHg)(min)(L)⁻¹. In a number of embodiments, the maximum total resistance of the device is no greater than 60 (mmHg)(min)(L)⁻¹

In a number of embodiments, the fiber bundle has a surface area in the range of 560-1165 cm². The diameter-to-length ratio of the fiber bundle may, for example, be in the range of 0.78-3.64 or in the range of 0.78 to 2.35. The priming volume may, for example, be no greater than 30 ml. The priming volume of the device may, for example, be in the range of 5-30 mL, 5-20 mL, 5-15 mL or 5-10 mL.

The device may, in a number of embodiments, achieve a carbon dioxide removal rate of at least 8.5 mL/min, or at least 12.2 mL/min, and an oxygenation range of 8.5 mL/min, or at least 12.2 mL/min, at a flow rate of 165 mL/min. In a number of embodiments, a normalized index of hemolysis (NIH) during operation of the device is less than 0.05 g/100 L.

In a number of embodiments, the plurality of hollow gas permeable fibers of the fiber bundle extend generally perpendicular to the direction of bulk flow of blood through the fiber bundle from the second end of the fiber bundle to the first end of the fiber bundle. The plurality of hollow gas permeable fibers may, for example, be formed in at least one generally cylindrical bundle. In a number of embodiments, the generally cylindrical bundle is formed from a plurality of layers of fiber fabric, each of the plurality of layers of fiber fabric comprising hollow gas permeable fibers. In a number of embodiments, bulk flow of blood through the fiber bundle is in a generally axial direction.

In a number of embodiments, the fiber bundle compartment includes an inlet manifold at a first end thereof, which is in fluid connection with the blood inlet, and an outlet manifold at a second end thereof, which is in fluid connection with the blood outlet. The (blood-contacting) wall of the inlet manifold may, for example, be filleted. A channel of the inlet manifold may extend generally tangentially from an axial end of the inlet manifold. The (blood-contacting) wall of the outlet manifold may also be filleted. In a number of embodiments, a channel of the outlet manifold extends from the inlet manifold at a position below an axis of the fiber bundle compartment with respect to the gravity vector.

In another aspect, a system includes a device hereof, a flow path to place the device in fluid connection with the circulatory system of the premature infant, and a source of sweep gas (that is, an oxygen-containing gas such as essentially pure oxygen). The flow path may, for example, include a cannulation system configured for cannulation of one or both umbilical arteries of the premature infant and a cannulation system configured for cannulation of the umbilical vein of the premature infant.

In a further aspect, a method of providing care to a premature infant includes placing a device hereof in fluid connection with the circulatory system of the premature infant. The device may, for example, be placed in fluid connection with the circulatory system of the premature infant via a cannulation system configured for cannulation of one or both umbilical arteries of the premature infant and a cannulation system configured for cannulation of the umbilical vein of the premature infant

The present devices, systems, and methods, along with the attributes and attendant advantages thereof, will best be appreciated and understood in view of the following detailed description taken in conjunction with the accompanying drawings.

### BRIEF DESCRIPTION OF THE DRAWINGS

FIG. 1A illustrates an isometric view of an embodiment of a hollow fiber membrane oxygenator hereof.

FIG. 1B illustrates a side view of the oxygenator of FIG. 1A.

FIG. 1C illustrates a side view of the oxygenator of FIG. 1A.

FIG. 1D illustrates a cross-sectional view of the oxygenator of FIG. 1A along section A-A as set forth in FIG. 1C.

FIG. 1E illustrates an exploded view of the stacking of fiber membranes to form a fiber bundle for use in an oxygenator hereof.

FIG. 1F illustrates a system hereof in which the oxygenator of FIG. 1A is placed in fluid connection with an infant's vasculature or circulatory system wherein flow of blood through the system is represented by arrows.

FIG. 2 illustrates a resistance apparatus used in studies of oxygenators hereof.

FIG. 3 illustrates schematically an embodiment of gas exchange circuit used in studies hereof.

FIG. 4A illustrates computational fluid dynamics (CFD) analysis results for 165 mL/min flow showing fluid velocity (m/s) through the fiber bundle of a device hereof.

FIG. 4B illustrates CFD analysis results for 165 mL/min flow showing streamlines of velocity magnitude (m/s) through a device hereof.

FIG. 4C illustrates CFD analysis results for 165 mL/min flow of velocity magnitude (m/s) through the blood inlet of a device hereof.

FIG. 4D illustrates CFD analysis results for 165 mL/min flow of velocity magnitude (m/s) through the blood outlet of a device hereof.

FIG. 5 illustrates a graph of elapsed time vs. In (hᵢ/h_{f}), wherein the slope of the linear relationship was used to determine the device resistance.

FIG. 6 illustrates plasma free hemoglobin (pfHb) concentration over time for experimental and control circuits, demonstrating that levels of pfHb did not statistically increase over time for either circuit (repeated measures ANOVA, p=0.24).

FIG. 7 illustrates in-vitro values of the CO₂ removal rate of the experimental HFM bundle compared to predictions of CO₂ removal by the model that accounts for the Haldane effect and a similar model that does not, wherein standard deviations are included.

FIG. 8 illustrates in-vitro values of the oxygenation rate of the experimental HFM bundle compared to predictions of oxygenation by the model that accounts for the Haldane effect and a similar mode that does not.

### DESCRIPTION

The present devices, systems, methods and compositions, along with the attributes and attendant advantages thereof, will best be appreciated and understood in view of the following description taken in conjunction with any accompanying drawings.

A number of embodiments hereof provide devices, systems and method for use in connection with a premature infant (for example, in connection with an artificial uterine environment) to achieve gas exchange.

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

As used herein and in the appended claims, the singular forms “a,” “an”, and “the” include plural references unless the context clearly dictates otherwise. Thus, for example, reference to “fiber bundle” includes a plurality of such fiber bundles and equivalents thereof known to those skilled in the art, and so forth, and reference to “the fiber bundle” is a reference to one or more such fiber bundles and equivalents thereof known to those skilled in the art, and so forth. Recitation of ranges of values herein are merely intended to serve as a shorthand method of referring individually to each separate value falling within the range. Unless otherwise indicated herein, each separate value as well as intermediate ranges are incorporated into the specification as if it were individually recited herein. All methods described herein can be performed in any suitable order unless otherwise indicated herein or otherwise clearly contraindicated by the text.

As used herein, the terms “approximately” and “generally” when used with respect to a value means within 10% or, more typically, within 5% thereof. As used herein, the term “generally cylindrical” (for example, as applied to a fiber bundle), refers to a component having an outer radius which varies by less than 10% (or less than 5%) from an average radius along the axis thereof.

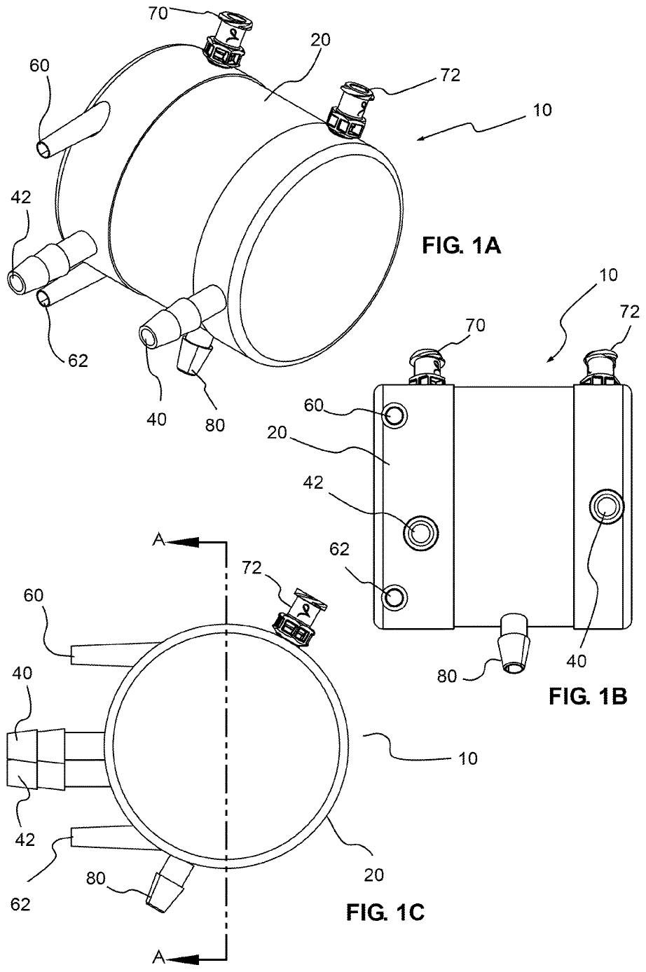

In a number of representative embodiments of studied, highly efficient hollow fiber membrane oxygenators (HFMOs) as illustrated, for example, in FIGS. 1A through 1D, and 1F, the oxygenator included a fiber bundle formed from representative polymethylpentene (PMP) fibers (OXYPLUS™, 3M™ MEMBRANA™). In a representative studied embodiment, the fiber bundle had a diameter of approximately 2.5 cm, a length of approximately 3.2 cm, a surface area of approximately 0.1 m², and a porosity of 0.48. The fiber bundles hereof may, for example, be a generally cylindrical bundles of hollow fiber membranes stacked in layers at, for example, 5-15 degree angles (as rotated around the axis thereof) to one another and aligned generally perpendicular to the principal direction of blood flow (that is, generally perpendicular to the axis A of fiber bundle compartment 22 and fiber bundle 100; see, FIGS. 1D and 1E) to maximize gas exchange.

In a number of representative studied embodiments, the fiber bundle was a generally cylindrical bundle of hollow fiber membranes stacked in layers at approximately 14 degree angles to one another. In that regard, the fibers were cut into round sheets and stacked at a 14 degree angle between adjacent sheets into a potting mold. In a number of embodiments, the fiber bundle is formed by stacking hollow fiber membrane fabric into a three-piece reusable mold made from Delrin® (a high-performance acetal resin also known as Polyoxymethylene). The mold/fixture helps to ensure consistency between all devices throughout the fabrication process.

The ends of the hollow fibers were potted into semi-circular gas manifold channels (a gas inlet manifold channel and a gas outlet manifold channel). A polyurethane or other glue may be injected into the mold by using centrifugal force generated by spinning the mold in a lathe. The glue binds all the fibers into the fiber bundle. The thickness of the potting glue may be readily determined to provide adequate mechanical support. In the studies hereof, the fibers were potted and molded around the periphery by centrifugally injecting a two-part polyurethane adhesive (Cas Chem, Bayonne, NJ) into the mold as described above. The mold was removed after the adhesive dried, and the potted fibers were exposed and tomed in a custom fixture to establish a common pathway between all fibers. The potted bundle was assembled into a main housing that creates separate blood and gas pathways. Aligning the hollow fibers generally perpendicular (for example, within no more 5 degrees from perpendicular or within no more than 2.5 degrees of perpendicular) to the axis of the fiber bundle can significantly decrease volume (that is, improve compactness) as compared to systems in which hollow fibers are generally parallel to the axis of the housing and bulk blood flow through the fiber bundle. This design allows for a highly compact extracorporeal life support device that closely mimics the native placenta in terms of priming volume, resistance, blood flow, and gas exchange performance.

The stacked-type fabrication method described above in connection with PMP fibers allows for a highly compact form factor with efficient gas exchange. This provides a technological advantage over other utilized hollow fiber membranes as the extremely premature infant is significantly smaller and more fragile than current pediatric ECMO patients. As described further below, the bundle housing was designed to generate fully developed flow through the bundle to prevent stasis and increase hemocompatibility. These design advantages result in a hollow fiber membrane gas exchanging device that is more comparable to the human placenta when compared to other devices. In a number of embodiments, a zwitterionic coating may be formed on all blood-contacting surfaces to increase the hemocompatibility of the device. In addition, in a number of embodiments, the device hereof enables the native vasculature to control flow through the device rather than requiring the use of a peristaltic or other pump.

As illustrated in FIGS. 1A through 1D, and 1F, a representative oxygenator 10 hereof includes a housing 20, A fiber bundle compartment 22 (see, for example, FIG. 1D) is formed within housing 20. Fiber bundle compartment 22 houses a fiber bundle 100 (see FIG. 1D) and provides a gas pathway designed to uniformly perfuse the gas side of fiber bundle 100 with a sweep gas which may be oxygen or a gas mixture including oxygen. A first end of fiber bundle compartment 22 includes an inlet blood manifold or volume 30 which is in fluid connection with a blood inlet 40 via which blood enters housing 20. A second end of fiber bundle compartment 22 includes outlet blood manifold or volume 32 which is in fluid connection with a blood outlet 42 via which blood exits housing 20. In a number of embodiments, the blood enters a first end of fiber bundle 100 from inlet blood manifold 30 and passes around the hollow fibers thereof. After passing through fiber bundle 100, blood exits oxygenator 10 via outlet blood manifold 32, which is in fluid connection with a second end of fiber bundle 100 and blood outlet 42.

The liquid/fluid flow path may be separated from the gas flow path through device 10 by abutment/sealing between (i) the periphery of the rear side of fiber bundle 100 and (ii) the periphery of the front side of fiber bundle 100 and fiber bundle compartment 22. The gas pathway in device 10 may, for example, be relatively simple. Gas flows in through a gas inlet port 60 into a channel on one side of fiber bundle 100 and out through a gas outlet port 62 in fluid connection with a channel on the other side of fiber bundle 100. See, for example, PCT International Publication No. WO 2019/143623, the disclosure of which is incorporated herein by reference.

Access to the infant vasculature system/circulatory system may, for example, be achieved via cannulation of both umbilical arteries. FIG. 1F illustrate a system 5 hereof in which a cannula/catheter system 200 may be used to connect to one or both of the infant umbilical arteries. Cannula system 200 is connected to blood inlet 40 of oxygenator 10 via tubing 210. Cannula/catheter system 202 may be used to connected to the infant umbilical vein. Cannula 210 is connected to blood outlet 42 of oxygenator 10 via tubing 212. Such an embodiment of a cannulation method eases the strain on the EPI's underdeveloped heart and mirrors the native placenta's vascular access route. Although flow of blood through system 5 may be driven solely by the fetal heart, in certain embodiments an optional pump may be provided in the flow path of system 5 as illustrated schematically in FIG. 1F.

In the illustrated embodiment of FIGS. 1A through 1D, and 1F, oxygenator 10 further includes ports 70 and 72 which function as both de-airing and pressure monitoring ports. Further, a drainage port 80 was provided in case there was an accumulation of condensation in the outlet sweep gas plenum.

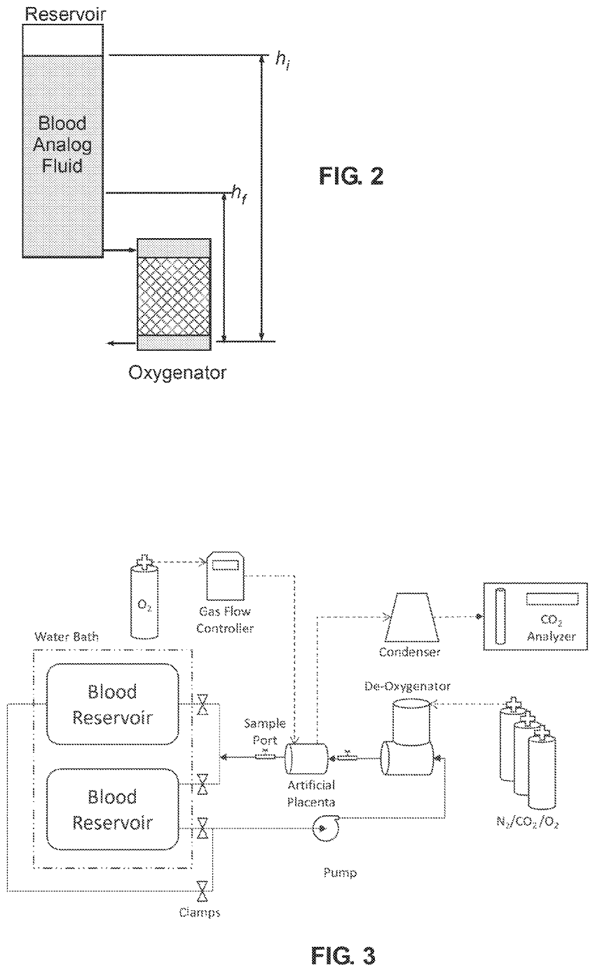

The design of the blood inlet and outlet manifolds 30, 32 of devices 10 hereof was optimized to reduce priming volume and provide a smooth blood pathway that produced fully developed blood flow without areas of stagnation or recirculation. In the studied embodiments, manifold diameter was based solely on the diameter of hollow fiber membrane bundle 100. Manifold depth (for example, 7 mm in a number of studied embodiments) was serially modified in models hereof until fully developed and straightened flow resulted at the bundle face. Manufacturing considerations also drove manifold depth as proper wall thickness is required to prevent material warping or damage. In the studied embodiments, inlet channel 40 (that is, the flow channel defined by the inner wall/diameter of blood inlet 40) of blood inlet manifold 30 was selected to be the largest inner diameter 3/16″ connector commercially available. The inlet flow channel was tangent to the deepest (that is, the axially outermost) portion of manifold 30 to prevent recirculation or fluid movement divergent from bulk flow (see, for example, FIGS. 4A through 4D). The outer wall or rim of the manifold was filleted (fillet F1 having radius of, for example, 6.35 mm in a number of embodiments; see FIGS. 4A and 4C) to direct flow from the inlet channel toward the face of fiber bundle 100. Between the manifold fillet and the inlet channel a right angle may be formed. Such a right angle would cause flow separation and recirculation that could result in excess hemolysis or clot formation if left unresolved. A fillet F2 (see FIGS. 4A and 4C) was thus added to this intersection and the radius (for example, 2.75 mm in a number of embodiments) was optimized to reduce the above-mentioned flow separation and recirculation. FIG. 4C illustrates fillet F2 as well as a portion of a similar oxygenator blood inlet manifold which include fillet F1 but does not include fillet F2.

As, for example, illustrated in FIG. 1D, in a number of embodiments, the flow conduit of blood inlet 40 enters manifold 40 in a generally radial direction relative to axis A of fiber bundle compartment 22 and fiber bundle 100 and is generally horizontally oriented (that is, generally perpendicular to the gravitation vector represented by arrow G) in the illustrated embodiment. Blood outlet 42 is oriented generally parallel to blood inlet 40 (that is, generally perpendicular to the gravitation vector represented by arrow G) but is offset downward from axis A (relative to the orientation of the gravity vector G). Such an offset of blood outlet 42 assists in preventing the settling of red blood cells under the force of gravity. In that regard, in some studies in which blood outlet 42 is not offset (that is, in which it extend generally radially and horizontally with respect to axis A and gravity vector G), red blood cells settle and become trapped below the outlet flow. By positioning the axis of blood outlet 42 below a centerline defined by axis A (toward or coincident with a position tangent to the bottom of outlet manifold 32) the bulk flow of the blood collects such blood cells and they are preventing from becoming a nidus for coagulation.

Once again, the oxygenators hereof are smaller than any oxygenator currently used in the field. The size of the oxygenator typically accounts for most of the surface area present in an ECMO circuit. Decreasing device surface area also mitigates the likelihood of circulatory overload, required anticoagulation to achieve therapeutic levels, and exposure of blood to foreign surface area. In addition, bundle geometry was configured to minimize device resistance. This allows circuit flow to be driven by the fetal heart and reduces heart afterload, curtailing chances of heart failure due to hypertension. In a number of embodiments, the requirements for the device include a priming volume less than 30 mL, a resistance less than 71 mmHg/L/min, a vCO₂ of at least 8.5 mL/min and an outlet hemoglobin saturation of 100% at a blood flow rate of 165 mL/min, and a normalized index of hemolysis (NIH) less than 0.05 g/100 L. A HFMO geometry that met these requirements was modeled, manufactured, and characterized in-vitro with gas exchange and hemolysis studies.

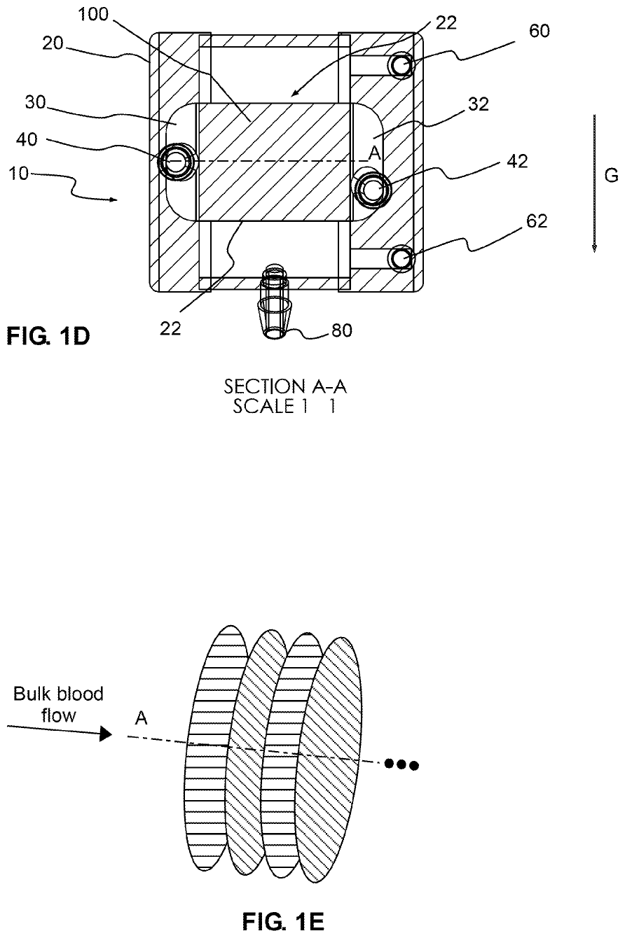

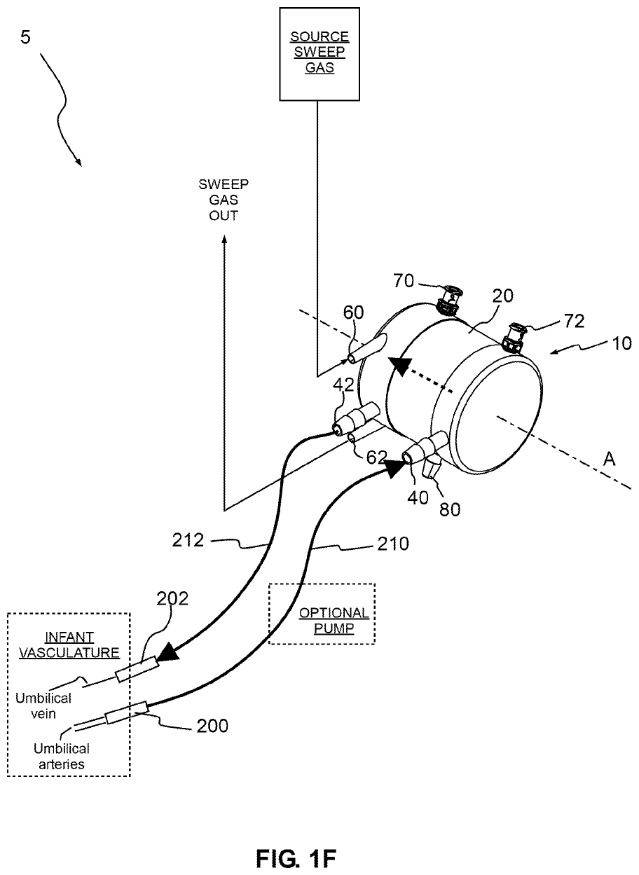

FIG. 2 illustrates an apparatus configuration for resistance studies hereof. In the apparatus of FIG. 2, fluid from the apparatus drains into a collection reservoir (not illustrated). Scissor lab jacks were used to keep the reservoir and device level. FIG. 3 provides a schematic illustration of a gas exchange circuit used in the studies hereof. In the illustrated circuit, clamps direct the recirculation of blood through the venous reservoir during conditioning. Once conditioning is complete, blood flow is directed through the circuit to the empty reservoir. During this single pass flow, gas flow via the de-oxygenator is stopped and pure oxygen sweep gas is flowed through the artificial placenta. Once the conditioned blood is depleted, the circuit is converted back to a recirculation circuit so blood can be conditioned to venous values.

In general, in a number of studies hereof, the design of the devices hereof was driven by priming volume, resistance, and gas exchange requirements of a preterm fetus. A goal of a number of embodiments of devices hereof is to treat a fetus as young as 22 weeks gestational and be able to provide support until the fetus is 28 weeks of gestational age. The maximum blood flow rate that the placenta experiences over this time period is approximately 165 mL/min. That blood flow rate was therefore targeted as the maximum requirement of the device. Additionally, the smallest blood volume of the placenta over this time period is approximately 32 mL and the smallest resistance of the placenta is 0.1 (mmHg)(min)(mL)⁻¹ or 100 (mmHg)(min)(L)⁻¹. If the device had a resistance greater than this value, it would be possible that younger and lighter infants would be unable to use the device safely (without an auxiliary/extracorporeal pumping mechanism). Therefore, that resistance was selected as the highest allowable resistance of the entire support circuit including flow path connecting the device to the premature infant (that is, for example, cannulas and tubing), With a total length of 36″ of 3/16″ tubing, two 3 mm diameter 5 cm long umbilical artery cannulas, and one 5 mm diameter and 5 cm long umbilical vein cannulas, the total resistance of the device could be, at maximum, 0.07 (mmHg)(min)(mL)⁻¹ or 71 (mmHg)(min)(L)⁻¹. This represents the greatest load that the device would be expected to provide. With parameters to define maximum device resistance, priming volume, and gas exchange, a device that could meet all three was designed. The gas exchange requirements of the device were determined by what the maximum oxygenation and CO₂ removal rates would be in the largest of infants (28 weeks). The determined gas exchange requirements at 165 mL/min flow were 8.5 mL/min for oxygenation and 8.5 mL/min for CO₂ removal.

Initially, a device capable of achieving the needed oxygen and CO₂ removal requirements was designed. A resistance of half of the maximum was targeted as there would be resistance associated with the inlet and outlet of the device. A total of 52 bundles were modeled, 16 of which met the gas exchange, resistance, and volume requirements. Those studied bundles varied is surface area in the range of 562-782 cm², in diameter-to-length ratio in the range of 1.2-3.6, in priming volume in the range of 5-7 mL. The indicated surface area is the cumulative surface area of the outsides surfaces of the hollow fibers in the fiber bundle.

Once it was determined that the device could meet the gas exchange requirements while exhibiting less than half the maximum resistance, it was decided to include a factor of safety. In that regard, detritus can be deposited or grow inside of the hollow fiber membrane bundle which reduces device efficiency. In the case of premature infants, removal and exchange of devices is more dangerous than in adults or older children as they are unable to produce their own blood and require transfusions. When a device is removed, the blood within the device is also removed. When a new device is put in place, a transfusion of blood is required to prime it. Increasing the number of transfusions an infant or other animal receives also increases the likelihood of transfusion reactions and resulting iatrogenesis. Therefore, the surface area of the 16 devices from the first design round were multiplied by approximately 1.5 by increasing the length of the device and leaving the original diameter unmodified.

After application of the factor of safety as described above, the bundles studied varied in surface areas over a range of 856-1165 cm², in diameter-to-length ratios varied over the range of 0.78-2.35, and the priming volumes varied over the range of 8-10 mL. The device with the smallest surface area was selected to be used as an initial prototype. In that regard, the greater the surface area a device has, the more likely blood will become damaged or deposit on the material surface. The prototype device had a length of 3.2 cm, a diameter of 2.5 cm, and a surface area of 856 cm². In a number of embodiments, the length of the fiber bundles hereof is in the range of approximately 1 cm to 3.5 cm and a diameter in the range of approximately 2.5 to 4 cm.

As discussed further below, the device design hereof (see, for example, FIGS. 1A through 1D, and FIGS. 4A through 4D) is able to achieve adequate gas exchange (for example, 12.7±0.9 mL/min) and resistance (for example, 51 mmHg/mL/min) at the maximum targeted blood flow rate (approximately 165 mL/min) while maintaining a physiologically relevant priming volume (for example, 15 mL of blood). Such results indicated that devices hereof are more physiologically comparable to the native placenta, and thus more clinically relevant, than other artificial placentas currently under study/development.

As described above, one of the goals of the studies of device hereof was to design and test a HFMO that mimics the resistance and gas exchange capabilities of a placenta from an EPI. This goal was achieved using a mathematical model of mass transfer and computational fluid dynamics verified with benchtop testing. The mathematical model used in the studies hereof is described further below. A representative HFMO device hereof was developed that has a surface area that is 33-88% smaller and a priming volume that is up to 81% smaller than devices utilized in other published studies.

Computations predicted a vCO₂ of 12.2 mL/min at 165 mL/min blood flow rate and an outlet saturation of 100%. CFD results (see, for example, FIG. 4A) showed uniform flow distribution throughout the bundle with axial flow having a coefficient of variation equal to 3.3%. Maximum shear was predicted to be 2.4 Pa and was located at the elbow of the outlet plenum. Minimum shear was predicted to be 0.02 Pa and was located in the main volume of the outlet plenum. Flow patterns showed no recirculation or separation in the plenums or bundle as illustrated in FIG. 4B.

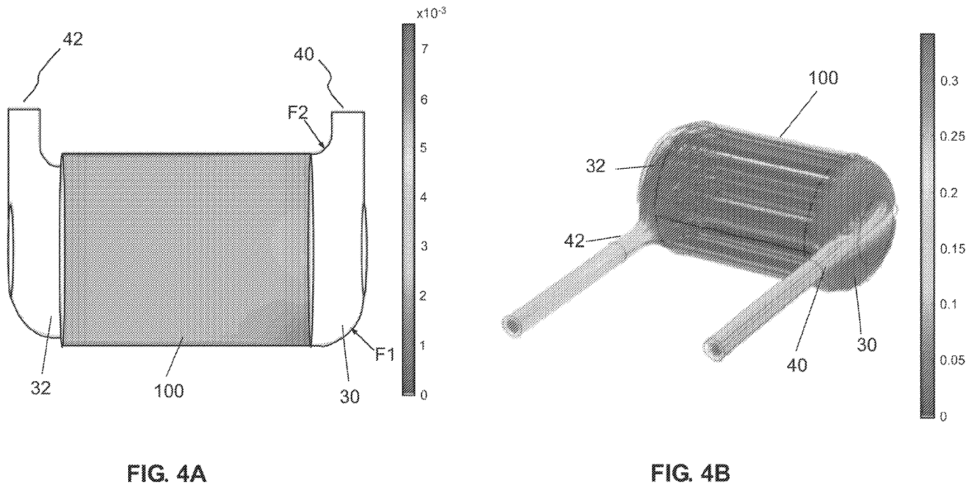

Ensuring that the artificial placenta circuit should have the same, or lower, resistance than the native placenta minimizes the potential for afterload induced congestive heart failure. Once again, the minimum placental resistance that a fetus experiences from mid-gestation to term is 100 mmHg/L/min. In a number of studies, the artificial placenta circuit is composed of three cannulas, two for the UAs and one for the UV, tubing, and the HFMO/oxygenator. To account for the resistance of the cannulas and tubing, the Hagen-Poiseuille equation was used to predict the pressure drop across two 9 Er arterial cannulas with a length of 5 cm in parallel, a 16 Fr venous cannula with a length of 5 cm, and 36″ of 3/16″ tubing. The inner diameters of the UAs and UV cannulas were estimated to be the same diameter of the respective arteries at 22 weeks GA. Total expected resistance of a circuit with this composition is 29 mmHg/L/min, leaving 71 mmHg/L/min available for the HEMO. The resistance of the device (as determined from the slope of linear relationship of the graph of elapsed time vs. ln(hᵢ/h_{f}) in FIG. 5) was 51±0 mmHg/L/min across all tested fluid column height changes. Total priming volume of the device was 15 mL. The measured resistance of the oxygenator was thus approximately 33% less (as determined by the equation V₁−V₂/(avg(V₁,V₂)) than the requirement of 71 mmHg/L/min, minimizing the chance of heart failure due to supraphysiologic resistance of the artificial placenta circuit. In that regard, a low resistance device minimizes fetal heart afterload and decreases the likelihood of hypertensive heart failure in an in-vivo setting.

Three sweep gas flow rates were tested to evaluate the point at which sweep gas independent vCO₂ is achieved. When the sweep gas flow rate to blood flow rate ratio is too low, the accumulation of CO₂ in the sweep gas reduces the gradient driving gas exchange, thereby reducing device efficiency. The regime in which this accumulation limits device efficiency is known as sweep gas dependent gas exchange. It has been shown that sweep gas independent vCO₂ is achieved when the gas flowrate is 40-60 times greater than the vCO₂. This benchmark was achieved within the a representative studied oxygenator hereof at the lowest tested sweep gas flow rate of 750 mL/min (60±3). Increasing sweep gas flowrate, once in the sweep gas independent regime, will not increase device efficiency. A one-way Analysis of Variance (ANOVA; which is a statistical formula used to compare variances across the means (or average) of different groups) showed that the vCO₂ was statistically equivalent between the three sweep gas flow rates tested (data not shown, p=0.22). Mean vCO₂ averaged over all three sweep gas flow rates was 12.7±0.9 mL/min at a blood flow rate of 163±2 mL/min (n==9). This is within 4% of computational predictions. Hemoglobin was completely oxygen saturated in all three cases (SO₂=99.4±0.4). The statistical equivalence of vCO₂ at all three sweep gas flow rates supports the evidence that sweep gas independence is achieved in the oxygenator at a sweep gas flow rate of 750 mL/min. In clinical practice targeted arterial conditions will be achieved using a N₂/O₂ sweep gas mixture.

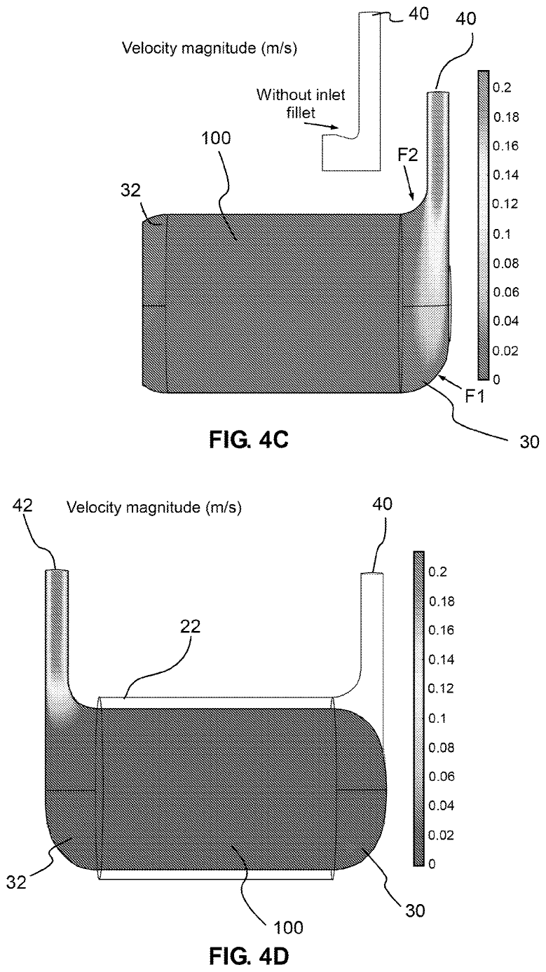

In a number of studies hereof, hemolysis was evaluated at a flow rate higher than the intended use of the oxygenator. Such studies were done to ensure that both pump rpm and flow rate were similar between the two circuits. Having statistically different flow rates or rpms between the two circuits would not support a 1-to-1 comparison between the experimental and control device. Despite running the circuits at a blood flow rate higher than the maximum intended use of the oxygenator, the generation of plasma free hemoglobin (pfHb) was not detected in either circuit. This result was expected as the root-mean-square shear stress within the device at this flow rate (0.8 Pa) is 35% smaller than the value that has been shown to significantly increase cellular and molecular mediators of coagulation. In addition, no material induced trauma was expected based on the relatively low surface area of the HFM bundle. It is logically predicted that, at the lower blood flow rate of 165 mL/min, the experimental and control circuits would not generate any measurable quantity of pfHb as shear stress within the circuit would decrease with blood flow rate. FIG. 6 shows pfHb over time for the control and experimental circuit. Levels of pfHb did not change over time according to a repeated measured ANOVA (p=0.24). The data of FIG. 6 demonstrates that no hemolysis was detected in either the control or experimental circuit over six hours. Therefore values of a normalized index of hemolysis (NIH) and a therapeutic index of hemolysis (TIH) could not be calculated. A phosphorylcholine coating may be provided to minimize the immune reaction to hydrophobic PMP fibers.

A potential limitation of the in-vitro analysis of the oxygenator is the use of adult bovine blood for hemolysis and gas exchange experiments. Pediatric patients treated with ECMO show trends of increasing hemolysis as patients decrease in age and weight. Fetal red blood cells (RBCs) are known to have a shorter lifespan, a larger size, less deformability, and are more fragile than adult RBCs. EPIs also typically suffer from anemia of prematurity resulting from a lack of maternal iron, a delayed EPO response, and phlebotomy losses from clinical testing. As a result of these factors, patients receive an increase in the number of adult blood transfusions correlating with a decrease in GA. The quantification of the effect that the composition of fetal blood may have on device generated hemolysis should therefore be considered. The lack of plasma free hemoglobin generation resulting from benchtop studies, however, provides assurance that the use of the oxygenator should not result in irreparable levels of hemolysis.

Adult hemoglobin (HbA) contains two alpha and two beta chains each associated with a heme group. Fetal hemoglobin (fHb) structurally differs from HbA as it contains two alpha and two gamma chains. Gamma chains have an increased affinity for oxygen compared to the Beta chains present in HbA. In-utero this adaptation allows fetuses to achieve adequate gas exchange despite the relatively low (compared to atmospheric) oxygen content of placental blood. An HFMO placed into fetal circulation could achieve higher rates of oxygenation, up until complete Hb saturation, than when used at the same blood flow rate in an adult. The oxygenation rate of the oxygenator devices hereof is not expected to increase in the fetal setting as it achieves 100% oxygen saturation in adult blood. CO₂ removal, however, may increase in the fetal setting as the increased affinity of fHb for oxygen results in an increased offloading of Hb bound CO₂ into plasma. In this manner the CO₂ concentration gradient between the sweep gas and blood would increase, resulting in increased vCO₂. In this manner, testing the oxygenators hereof in adult blood represents a hypothetical worst-case scenario where the entire circulating fHb volume of the fetus has been replaced with HbA. The computationally confirmed uniform blood flow through the representative PMP stacked type hollow fiber membrane bundles and devices hereof thus achieved highly efficient gas exchange.

As described above, mathematical modeling of device design hereof was utilized to improve device hemocompatibility and efficiency. A previously published method for mathematical modelling to predict CO₂ removal in hollow fiber membrane bundles was modified to include an empirical representation of the Haldane effect. In a number of studies of the model hereof, the predictive capabilities of both models were compared to experimental data gathered from a fiber bundle of 7.9 cm in length and 4.4 cm in diameter. The CO₂ removal rate predictions of the model hereof including the Haldane effect reduced the percent error between experimental data and mathematical predictions by up to 16% compared to a similar model without consideration of the Haldane effect. Improving the predictive capabilities of computational fluid dynamics for the design of hollow fiber membrane bundles reduces the monetary and manpower expenses involved in designing and testing such devices.

As described above, the gas exchanging circuit component of ECMO or ECCO₂R therapy is a structure composed of microporous hollow fiber membranes (HFM) woven into sheets and folded into bundle structures (HFM bundle); blood flows through the bundle around the fibers while a sweep gas, usually pure O₂, flows through the fiber lumens. The juxtaposition of pure oxygen gas and venous blood creates a concentration gradient, causing O₂ to diffuse from the sweep gas across the membrane and into the blood and CO₂ to diffuse across the membrane from the blood to the sweep gas. The efficiency of a HFM bundles can be refined by iteratively modifying bundle characteristics, performing in-vitro testing, and comparing experimental results. This trial-and-error method is costly in terms of money, materials, and manpower. Computational fluid dynamics or CFD has thus evolved to be the primary method by which researchers design and compare HFM bundles before prototyping and testing.

A CFD model that predicts oxygenation rates in blood based on a dimensionless mass transfer correlation between the Sherwood, Schmidt, and Reynold's numbers has been developed. L. Mockros, R. J. Leonard, Compact cross-flow tubular oxygenators, Trans. Am. Soc. Artif. Intern, Organs. 31 (1985) 628-633, the disclosure of which is incorporated herein by reference. The correlation includes two empirical constants that were determined experimentally in water. To relate the mass transfer correlation developed in a simple fluid to blood, the differences in oxygen storage within water and blood had to be considered. Water carries oxygen only in a dissolved form that follows Henry's law. Blood carries oxygen dissolved in the plasma as well as bound to hemoglobin in the form of oxyhemoglobin. An effective diffusivity was included in the Schmidt number to account for the convection of oxyhemoglobin. The effect of oxyhemoglobin on the solubility of oxygen into the blood was accounted for using the slope of the oxygen-dissociation curve in the Sherwood and Schmidt numbers. This curve represents the change in total blood oxygen content which occurs with changing oxygen partial pressure.

The methodology was furthered by creating a dimensionless mass transfer correlation for both oxygen and carbon dioxide that collapsed onto one universal curve. R. G. Svitek, W. J. Federspiel, A Mathematical Model to Predict CO₂ Removal in Hollow Fiber Membrane Oxygenators, Annals of Biomedical Engineering, 36 (2008) 992-1003, the disclosure of which is incorporated herein by reference. This was done by utilizing the oxygenation relationships defined by the initial model and developing additional diffusivity and solubility relationships to account for the differences in CO₂ storage between water and blood. Carbon dioxide is also only carried by water in a dissolved form that follows Henry's law while CO₂ is stored in the blood in three ways: as bicarbonate (70%), bound to hemoglobin (23%), and dissolved in the plasma (7%). An effective diffusivity in the Schmidt number accounts for the convection of carbon dioxide carried as bicarbonate and as protonated hemoglobin. Within the Sherwood number a facilitated diffusivity accounts for the diffusion of bicarbonate in addition to CO₂ dissolved in the plasma. This facilitated diffusivity is not required for the oxygenation model as oxyhemoglobin only exists within an RBC and is therefore only carried by convection. The effect of protonated hemoglobin and bicarbonate on the solubility of CO₂ into the blood was accounted for using the slope of the CO₂ dissociation curve in both the Sherwood and Schmidt numbers. That curve represents the change in total blood carbon dioxide content which occurs with changing carbon dioxide partial pressure.

Published experimental data were used as the experimental values in calculations of percent error. A. G. May, R. G. Jeffries, B. J. Frankowski, G. W. Burgreen, W. J. Federspiel, Bench Validation of a Compact Low-Flow CO₂ Removal Device, Intensive Care Medicine Experimental. 6 (2018), the disclosure of which is incorporated herein by reference. The range of blood flow rates tested in this publication are relative for ECCO₂R where CO₂ removal is the goal of therapy. The characteristics of the HFM bundle tested in that publication are listed in Table 1 below. Also presented is the predicted CO₂ removal and oxygenation rates of the experimental HEM bundle mathematically calculated by the previous iteration of the model hereof, wherein the Haldane effect is not accounted for.

TABLE 1
Characteristics of ModBLAS HFM bundle

| Parameter | Description | Value | |
|---|---|---|---|
| A | Cross sectional area of ModELAS | 16 | cm² |
| | HFM bundle | | |
| αᵥ | Surface area to volume ratio of | 55 | cm⁻¹ |
| | ModELAS HFM bundle | | |
| A_{f} | Active fiber surface area of | 6700 | cm² |
| | ModELAS HFM bundle | | |
| d_{f} | Outer diameter of a single | 0.038 | cm |
| | OXPLUS ™, Membrana ™ | | |
| | PMP fiber | | |
| E | Porosity of ModELAS HFM | 0.48 | |
| | bundle | | |

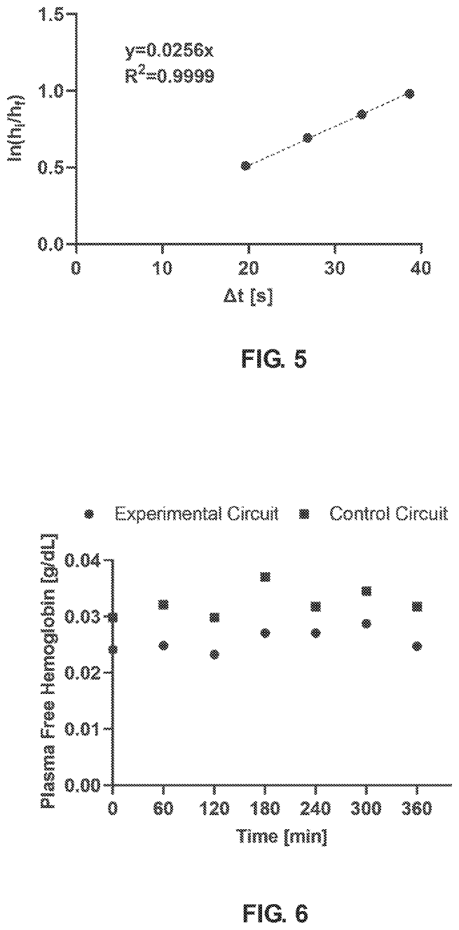

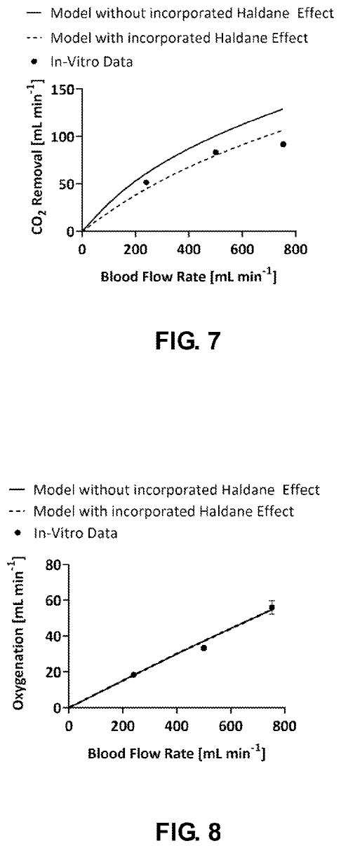

The previous iteration of the mathematical model, without the Haldane effect, produced a predicted CO₂ removal rate within 16% error at a blood flow rate of 240 mL min⁻¹, 18% error at a blood flow rate of 500 mL min⁻¹, and 30% error at a blood flow rate of 753 mL min⁻¹. (See FIG. 7) The proposed mathematical model, including the Haldane effect, predicted a CO₂ removal rate within 15%, 4%, and 14% error, respectively. FIG. 8 illustrates in-vitro values of the oxygenation rate of the experimental HFM bundle compared to predictions of oxygenation by the model that accounts for the Haldane effect and a similar mode that does not. Given the identity in the manner of such predictions of oxygenation, those values are identical.

The inclusion of the Haldane effect into the mathematical model melded the CO₂ removal and oxygenation models as the mass balances became a paired set of first order differential equations. However, only the CO₂ model underwent modifications to enable the output of the oxygenation model, O₂ content of the blood, to become an input variable for the CO₂ mass balance. The oxygenation model was therefore unaffected by the change and both the code that incorporates the Haldane effect and the one that does not produce the same values of oxygenation at the same blood flow rates. At 240 mL mind blood flow rate the models predicted an oxygenation rate within 2% error, at 500 mL min⁻¹ the prediction was within 10% error, and at 740 mL min⁻¹ the prediction was within 2% error.

With the acceptance and growing clinical use of ECMO and ECCO₂R the need for more compact, efficient, and user-friendly systems has become a focus for medical product development. The performance of competing oxygenator designs can be quantitatively compared after manufacturing and benchtop testing. Researchers have turned to the use of computational fluid dynamics to evaluate oxygenator designs as this trial-and-error method requires a great deal of resources and manpower. The modifications of CFD models described herein address a limitation of previously published oxygenation and carbon dioxide removal mathematical models. As described above, the Haldane effect was mathematically incorporated into these models via an empirical fit of the CO₂ dissociation curve. This coupled the previously univariate models into a single multivariate model. The inclusion of the Haldane effect increased the accuracy of the CO₂ removal predictions made by the model when compared to the same mathematical model with a static CO₂ dissociation curve, (that is, without the Haldane effect). The improvement in the CO₂ removal predictions is associated with the buffering capacity of single red blood cells.

The constant curve used by the model that does not account for the Haldane effect assumes blood is at an oxygen saturation of 100% and a Hb=15 g (dL blood)⁻¹. Testing conditions for oxygenators, however, typically dictate venous oxygen saturation to be 65% and blood hemoglobin to be 12 g (dL blood)⁻¹. Oxyhemoglobin is a stronger acid than both unbound hemoglobin and protonated hemoglobin. As hemoglobin becomes oxygenated, protonated hemoglobin is forced to dissociate into unbound hemoglobin and a proton. Carbaminohemoglobin is also forced to dissociate, displacing additional intraerythrocytic CO₂ into the plasma. Therefore, at the same total concentration of CO₂ in the blood, a system with a greater oxygen saturation will store a greater percentage of CO₂ within the plasma rather than within the red blood cell (that is, the Haldane effect). This creates a larger partial pressure gradient to drive mass transfer between the blood and sweep gas, effectively increasing the rate of CO₂ exchange achieved by the HFM bundle.

At the same pH, partial pressure of CO₂, and SO₂, a system with a higher hemoglobin, 15 g (dL blood) will have a smaller total concentration of CO₂ than a system with a lower hemoglobin, 12 g (dL blood)⁻¹. As hemoglobin increases there is a resulting decrease in overall space in the plasma, an increase in the pH of blood, and a decrease of bicarbonate ions. It has been shown, however, that increased hemoglobin results in increased CO₂ removal rates in identical oxygenators at the same blood flow rate, inlet pH, inlet partial pressure of CO₂, and inlet SO₂. The increased CO₂ removal but decreased overall CO₂ content seems counterintuitive, however one is still seeing the Haldane effect at work. In a system with a Hb of 15 g (dL blood)⁻¹ at a saturation of 65%, a total of 9.8 g (dL blood)⁻¹ of Hb are present in the form of oxyhemoglobin. In a system with a Hb of 12 g (dL blood)⁻¹ at a saturation of 65%, a total of 7.8 g (dL blood)⁻¹ of Hb are present in the form of oxyhemoglobin. The increased presence of oxyhemoglobin forces a greater amount of protonated hemoglobin and carbaminohemoglobin to dissociate. Even though the overall content of CO₂ in the 15 g (dL blood)⁻¹ of Hb system is smaller than the alternative, a greater amount of it is stored within the plasma and there is a higher partial pressure gradient present to drive CO₂ exchange mass transfer. In addition, the greater presence of oxyhemoglobin and the increased area available for the chloride shift results in an acceleration of the dissociation of the various forms of CO₂ from hemoglobin. This is also compounded by a greater presence of Hb which provides an additional increase to the whole blood pH. There is therefore a greater change in total CO₂ content per change in partial pressure of CO₂ compared to a system with less Hb. Mathematically, the CO₂ dissociation curve for a system with a higher Hb value will therefore have a greater slope at any one partial pressure of CO₂ than a system with a smaller concentration of Hb.

In this manner, utilizing the model without the Haldane effect results in a CO₂ dissociation curve that is right shifted and has greater slope than is present in the physiological system. This ultimately results in an overestimation of the CO₂ gradient that drives mass transfer and therefore the CO₂ removal capabilities of a modeled HFM bundle. The empirical equations utilized to incorporate the Haldane effect are limited in accuracy to the range of conditions over which the physiologic data was gathered and the relationships were established. The assumption of the CO₂ dissociation curve's linearity on logarithmic co-ordinates (Eq. 2 below; in the Experimental section) has been verified between for CO₂ partial pressure values of 20-80 mmHg by plotting human data. Eq. 19 below was defined in the 1920s, via a fit of experimental data, between CO₂ partial pressure values of 30-60 mmHg. As values stray from these ranges the predictive capabilities of the model may become less accurate. CO₂ partial pressure values experienced during the collection of the presented in-vitro data ranged from 9-45 mmHg. CO₂ partial pressure values that were the farthest outside of the validated empirical range were experienced at a blood flow rate of 250 mL min⁻¹. This correlates to the highest experienced percent error, 16%, between code predictions and experimental data. The code without the Haldane Effect also experienced a 16% error at a blood flow rate of 250 mL min⁻¹, but the highest experienced percent error was 30% at a blood flow rate of 753 mL min⁻¹. The inclusion of the Haldane Effect may therefore still provide more accurate predictions of CO₂ removal, when used outside of the validated ranges, when compared to the code without the Haldane effect.

### Experimental. Materials and Methods

Methods Underlying Development of Model Including Haldane Effect. The following analyses applies to a cylindrical axial fiber bundle with blood flow down the axis of the cylinder. A similar analysis can be done for annular fiber bundles with radial blood flow. See R. G. Svitek, W. J. Federspiel, A Mathematical Model to Predict CO₂ Removal in Hollow Fiber Membrane Oxygenators, Annals of Biomedical Engineering. 36 (2008) 992-1003.

Steady State Mass Balance of Carbon Dioxide. The steady state mass balance for CO₂ along the length of the bundle is:

$${Q}_{b} \frac{{dC}_{{CO}_{2}}}{dz} = A ( {a}_{v} ) {k}_{{CO}_{2}} ( {P}_{{CO}_{2} , b} - \overline{{P}_{{CO}_{2} , g}} ) \qquad [ 1 ]$$

where Q_{b} represents the blood flow rate, C_{CO}_{₂} represents the total concentration of CO₂ in blood, A is the area of the bundle perpendicular to blood flow (i.e. frontal area), aᵥ is the surface area to volume ratio of the bundle, k_{CO}_{₂} is the mass transport coefficient of CO₂, P_{CO}_{₂}_{,b} is the partial pressure of CO₂ in the blood, and P_{CO}_{₂}_{,g} is the partial pressure of CO₂ in the sweep gas. P_{CO}_{₂}_{,g} is typically low compared to P_{CO}_{₂}_{,b}, therefore the average of P_{CO}_{₂}_{,g} between gas flow inlet and outlet can be used.

The total concentration of CO₂ in the blood is represented as a mathematical fit of the CO₂ dissociation curve in the form [5]:

$${C}_{{CO}_{2}} = {qP}_{{CO}_{2} , b}^{t} \qquad [ 2 ]$$

where q and i are regression parameters dependent on the oxygen content of the blood. The regression parameters are derived below under the section subheading Incorporating the Haldane Effect.

Substituting Eq. 2 into Eq. 1 results in an equation of the form:

$${Q}_{b} qt ( {P}_{{CO}_{2} b}^{t - 1} ) \frac{{dP}_{{CO}_{2 , b}}}{dz} = - A ( {a}_{v} ) {k}_{{CO}_{2}} ( {P}_{{CO}_{2} , b} - \overline{{P}_{{CO}_{2} , g}} ) \qquad [ 3 ]$$

The mass transport coefficient, k_{CO}_{₂}, is a constant that relates mass transfer rate, mass transfer area, and the difference in partial pressure gradient that drives the movement of CO₂ from the sweep gas to the blood. The mass transport coefficient of CO₂ in blood can be determined from an analogous heat transfer correlation for flow perpendicular to a bundle of tubes in the form:

Sh=aRe^{b}Sc^{1/3} [4]

The Sherwood number, Sh, relates the ratio of convective mass transfer to the rate of diffusive mass transport. The Reynold's number, Re, is a ratio of inertial to viscous forces, and the Schmidt number, Sc, is the ratio of momentum to mass diffusivity. The coefficients a and b are dependent on the geometry of the HFM bundle and can be found in Table 2 below.

The Reynold's number describing flow conditions of a fluid within a packed bed takes the general form:

$$Re = \frac{{V}_{0}}{γ ψ a} \qquad [ 5 ]$$

where V₀ is the superficial velocity through the HFM bundle and γ is the fluid viscosity. Superficial velocity is a hypothetical fluid flow that is calculated by dividing the volumetric flow rate of fluid through the bundle by the cross-sectional area of the HFM bundle. The characteristic length, 1/ψa, considers a correction factor for the geometry of the packing the bed, ψ=0.91, and the surface area of the fibers per unit volume of the bundle:

$$a = \frac{6 ( 1 - E )}{{d}_{p}} \qquad [ 6 ]$$

where E is the bundle porosity and dₚ is the particle diameter. For cylindrical particles, the HFM fibers, the particle diameter is expressed as:

dₚ=0.567\sqrt{A_{f}} [7]

where A_{f} is the total surface area of the gas exchanging portion of the hollow fiber membranes.

The Sherwood number describing the flux of a gas into a fluid takes the general form:

$$Sh = \frac{kL}{α D} \qquad [ 8 ]$$

where k is the mass transport coefficient of the gaseous species, L is the characteristic length of the system, a is the solubility of the gas in the fluid, and D is the diffusivity of the gas into the fluid. For the flux of CO₂ into blood, the mass transport coefficient is an unknown and equal to k_{CO}_{₂}, the characteristic length is the outer diameter of a single fiber, d_{f}, and the solubility of CO₂ into blood is known and represented as a_{CO}_{₂}. The diffusivity, D, must consider the diffusion of CO₂ dissolved in the plasma and the diffusion of CO₂ stored as bicarbonate. This value will be referred to as the facilitated diffusivity and is represented mathematically by:

$${D}_{f} = {D}_{{CO}_{2}} + \frac{{D}_{{HCO}_{3}}}{{α}_{{CO}_{2}}} \frac{δ {C}_{{HCO}_{3}}}{δ {P}_{{CO}_{2} , b}} \qquad [ 9 ]$$

where D_{CO}_{₂} is the diffusivity of CO₂ in blood, D_{HCO}_{₃} is the diffusivity of bicarbonate in blood, a_{CO}_{₂} is the solubility of CO₂ in blood, and dC_{HCO}_{₃}/dP_{CO}_{_{2,b}} is the change in bicarbonate ion concentration with respect to partial pressure of CO₂ in the blood. dC_{HCO}_{₃}/dP_{CO}_{_{2,b}} is the slope of the CO₂ dissociation curve, Eq. 2, as the majority of carbon dioxide in the blood is stored as bicarbonate.

The Schmidt number takes the dimensionless form:

$$c = \frac{{ν}_{b}}{D} \qquad [ 10 ]$$

where v_{b} is the kinematic viscosity of blood. The diffusivity, D, must account for the convection of CO₂ stored as carbaminohemoglobin and bicarbonate. This value will be referred to as the effective diffusivity, D_{eff,CO}_{₂}, and is represented mathematically by:

$${D}_{e ff , {CO}_{2}} = \frac{{D}_{f}}{1 + \frac{1 δ {C}_{{HCO}_{3}}}{{α}_{{CO}_{2}} δ {P}_{{CO}_{2} , b}}} \qquad [ 11 ]$$

Substituting Eqs. 5 through 11 into the dimensionless correlation of Eq. 4 and rearranging to solve for the mass transport coefficient results in the mathematical equation:

$${k}_{{CO}_{2}} = \frac{{α}_{{CO}_{2}} {D}_{f} a R {e}^{b} S {c}^{1 / 3}}{{d}_{f}} \qquad [ 12 ]$$

Blood oxygen saturation is solved for using a steady state mass balance on the HFM bundle with oxygen as the species of interest.

Steady State Mass Balance of Oxygen. The steady state mass balance for O₂ along the length of the bundle is:

$${Q}_{b} \frac{{dC}_{{O}_{2}}}{dz} = A ( {a}_{v} ) {k}_{{O}_{2}} ( {P}_{{O}_{2} , b} - \overline{{P}_{{O}_{2} , g}} ) \qquad [ 13 ]$$

where C_{O}_{₂} represents the total concentration of O₂ in blood, k_{O}_{₂} is the mass transport coefficient of O₂, P_{O}_{₂}_{,b} is the partial pressure of oxygen in the blood, and P_{O}_{₂}_{,g} is the partial pressure of O₂ in the sweep gas. P_{O}_{₂}_{,g} is ideally high compared to P_{O}_{₂}_{,b}, therefore the average of P_{O}_{₂}_{,g} can be used.

The total concentration of O₂ in blood is a combination of oxygen dissolved in the plasma and bound to hemoglobin. This can mathematically be represented as:

C_{O}_{₂}=a_{O}_{₂}P_{O}_{₂}_{,b}+C_{T}HbSO₂ [14]

where a_{O}_{₂} is the solubility of O₂ in blood, C_{T} is the oxygen binding capacity of hemoglobin, Hb is the total hemoglobin level in the blood, and SO₂ is the percent of hemoglobin present in the form of oxyhemoglobin. Substituting the derivative of Eq. 14 into Eq. 13 gives:

$${Q}_{b} ( {α}_{{O}_{2}} + {C}_{T} \frac{{d SO}_{2}}{{dP}_{{O}_{2} , b}} ) \frac{{dP}_{{O}_{2} , b}}{dz} = A ( {a}_{v} ) {k}_{{O}_{2}} ( {P}_{{O}_{2} , b} - \overline{{P}_{{O}_{2} , g}} ) \qquad [ 15 ]$$

SO₂ is a function of the partial pressure of oxygen in blood, approximated well by the Hill equation:

$${SO}_{2} = \frac{{( {P}_{{O}_{2} , b} / {P}_{50} )}^{n}}{1 + {( {P}_{{O}_{2} , b} / {P}_{50} )}^{n}} \qquad [ 16 ]$$

where n and P₅₀ are constants dependent on the age and species of animal blood being tested and can be found in Table 2 below.

Eq. 4 can also be used to derive the mass transport coefficient of O₂, however the appropriate values for oxygen must be substituted into the general dimensionless values of the Reynolds, Sherwood, and Schmidt numbers. The Reynold's number, Eq. 5, applies for both the CO₂ and O₂ mass balance as it is not dependent on any gaseous species-specific values. For the flux of O₂ into blood, the mass transport coefficient in the Sherwood number is an unknown and equal to k_{O}_{₂}, the characteristic length remains as d_{f}, and the solubility of O₂ into blood is known and represented as a_{O}_{₂}. For oxygenation there is no facilitated diffusivity as the oxyhemoglobin is contained within the red blood cell and is transported only by convection. Therefore, the diffusivity, D, is simply the diffusivity of oxygen in blood, D_{O}_{₂}.

For the Schmidt number the viscosity remains the same, v_{b}, and an effective diffusivity must also be defined as the convection of the oxygen bound to hemoglobin must be considered. This is done by using the slope of the Hill equation, Eq.16, to approximate the slope of the oxyhemoglobin dissociation curve:

$${D}_{eff , {O}_{2}} = \frac{{D}_{{O}_{2}}}{1 + \frac{{C}_{T} δ {C}_{{O}_{2}}}{{α}_{{O}_{2}} δ {P}_{{O}_{2}}}} \qquad [ 17 ]$$

δC_{O}_{₂}/δP_{O}_{₂} is equivalent to the slope of Eq. 14.

Using Eqs. 5 through 8, 10, 14, 16, and 17 the mass transport coefficient, k_{O}_{₂}, becomes:

$${k}_{{O}_{2}} = \frac{{α}_{{O}_{2}} {D}_{{O}_{2}} a R {e}^{b} {Sc}^{1 / 3}}{{d}_{f}} \qquad [ 18 ]$$

Incorporating the Haldane Effect: As previously stated, the CO₂ dissociation curve can be represented by Eq. 2:

$${C}_{{CO}_{2}} = {qP}_{{CO}_{2} , b}^{t}$$

This equation is derived from a linear fit of any whole blood CO₂ dissociation curve when plotted on logarithmic coordinates. The mathematical model that does not include the Haldane effect assumed a constant q and t value to define the CO₂ dissociation curve throughout the entirety of the bundle. While this assumption greatly simplifies the mathematical calculations made within the model, it does not accurately reflect the compensatory mechanisms blood uses to achieve efficient CO₂ removal. It is within this section that an iteratively updating CO₂ dissociation curve will be included into the model to incorporate the Haldane effect.

It has been demonstrated that between the CO₂ partial pressure values of 30 mmHg and 60 mmHg the slope of the CO₂ dissociation curve is linearly related to the oxygen capacity of the blood being tested:

C_{CO}_{₂}(P_{CO}_{₂}=60)−C_{CO}_{₂}(P_{CO}_{₂}=30)=0.334(O₂cap)+6.3 [19]

This can then be related to hemoglobin concentration as it is known that 1.36 mL of oxygen combines with 1 gram of hemoglobin:

C_{CO}_{₂}(P_{CO}_{₂}=60)−C_{CO}_{₂}(P_{CO}_{₂}=30)=0.4542(Hb)+6.3 [20]

Using Eq. 2 in the definition of the slope of a linear function and substituting it for the left hand size of Eq. 20 results in the mathematical equality:

$${{C}_{C {O}_{2}} ( \frac{60}{{P}_{C {O}_{2 , b}}} )}^{t} - {{C}_{C {O}_{2}} ( \frac{30}{{P}_{C {O}_{2} , b}} )}^{t} = 0.4542 ( Hb ) + 6.3 \qquad [ 21 ]$$

which can then be solved numerically if an ordered pair of (P_{CO}_{₂}_{,b}, C_{CO}_{₂}) is known.

The initial value condition of P_{CO}_{₂}_{,b} is obtained from a blood gas analyzer or set of target venous conditions. In this model pH is considered to be constant as the percent increase from typical venous to arterial pH is 0.5%. An empirical mathematical equation relating these values has been established.

$${C}_{C {O}_{2}} = 2.2 \; 26 {HCO}_{3}^{-} [ 1 - \frac{0.02924 ( H b )}{( 2244 - 0422 {SO}_{2} ) ( 8.74 - pH )} ] \qquad [ 22 ]$$

where

$${HCO}_{3}^{-}$$

is the amount of carbon dioxide present in the blood in the bicarbonate form and SO₂ is a function of P_{O}_{₂}_{,b} according to Eq. 16. Bicarbonate serves as a buffer to the acidic presence of CO₂ in the plasma to maintain a physiologically safe blood pH. This acid-base homeostatic mechanism can be represented by the Henderson-Hasselbach equation in the form:

$${HCO}_{3}^{-} = 0.0301 {P}_{C {O}_{2} , b} ( 1 + 1 {0}^{p H - 6.10} ) \qquad [ 23 ]$$

Now with a single known value of P_{CO}_{₂}_{,b}, C_{CO}_{₂} can be calculated and Eq. 21 can be solved for the value of t.

Eq. 3 can be rewritten as:

$$= \frac{{C}_{{CO}_{2}}}{{P}_{{CO}_{2} , b}^{t}} \qquad [ 24 ]$$

and q can be solved for with the now known values of C_{CO}_{₂}, P_{CO}_{₂}_{,b} and S_{O}_{₂}, and

With Eqs. 19-24, the values of q and t are functions of P_{CO}_{₂}_{,b} and S_{O}_{₂}, and change accordingly as blood traverses through the HFM bundle. With the inclusion of S_{O}_{₂} in the mass balance for carbon dioxide, Eq. 1 and 13 become coupled first order differential equations that can be solved numerically. The initial values of S_{O}_{₂} and P_{CO}_{₂}_{,b} are directly measured from the blood entering the ECCO₂R device or a set of targeted venous conditions. The physical constants for O₂ and CO₂ are listed in Table 2. The above system of first order differential equations was solved in the MATLAB® programming platform (Math Works of Natick, MA) using the Runge-Kutta fourth order method.

TABLE 2
Physical Constants for O₂ and CO₂

| Parameter | Description | Value |
|---|---|---|
| P_{co2,b} | Value of P_{co2} in the blood | 45 mmHg |
| (z = 0) | entering the HFM bundle | |
| P_{o2,b} | Initial value of Pₒ₂ in the | 36 mmHg |
| (z = 0) | blood entering the HFM | |
| | bundle | |
| P_{c02,g} | Average value of P_{co2} in | 4 mmHg |
| | the sweep gas | |
| P_{o2,g} | Average value of Pₒ₂ in | 700 mmHg |
| | the sweep gas | |
| Hb | Hemoglobin of blood | 12 g (dL blood)⁻¹ |
| pH | pH of blood entering the | 7.4 |
| | HFM bundle | |
| P₅₀ | Pₒ₂ at 50% Hb saturation | 29 mmHg |
| | for adult bovine blood | |
| n | Hill parameter for adult | 2.85 |
| | bovine blood | |
| y | Kinematic viscosity of | 0.023 cm²s⁻¹ |
| | blood | |
| a_{CO2} | Solubility of CO₂ in | 6.62e−4 |
| | blood | (mL CO₂) |
| | | (mmHg) |
| | | (mL blood)⁻¹ |
| a_{O2} | Solubility of O₂ in | 3e−2 |
| | blood | (mL O₂) |
| | | (mmHg) |
| | | (mL blood)⁻¹ |
| D_{CO2} | Diffusivity of CO₂ in | 7.39e−6 cm²s⁻¹ |
| | blood | |
| D_{HCO3} | Diffusivity of | 4.62e−6 cm²s⁻¹ |
| | bicarbonate in blood | |
| D_{O2} | Diffusivity of O₂ in | 1.8e-5 cm2s-1 |
| | blood | |
| C_{T} | Binding capacity of | 1.34 mL O₂ |
| | hemoglobin | (g Hb)⁻¹ |
| Q_{b} | Blood flowrate | 0-600 (mL |
| | | blood) min⁻¹ |
| a | Measured coefficient | 0.54 |
| | for Eqs. 4, 12, and 18 | |
| b | Measured coefficient | 0.42 |
| | for Eqs. 4, 12, and 18 | |

Computational Design and Flow Evaluation. Bundles of varying geometries were computationally evaluated for gas exchange efficiency, priming volume, and pressure drop prior to prototype manufacturing. Previously published oxygenation and CO₂ removal mass transfer correlations modified to incorporate the Haldane Effect were used to predict the gas exchange capabilities of 52 potential geometries. The Haldane effect and such computations are described above. Of the 11 geometries that met both gas exchange and resistance requirements (see Table 3 below), the device with the lowest surface area was selected before further study and manufacture. The HFMO includes polymethylpentene (PMP) fibers (OXYPLUS™, 3M™ MEMBRANA™) and has a diameter of 2.5 cm, a length of 3.2 cm, a surface area of ~0.1 m² (FIG. 1), and a porosity of 0.48. Computational fluid dynamic analysis of the flow distribution in the device was completed using the Free and Porous Media Flow physics of COMSOL Multiphysics (COMSOL INC., Stockholm, Sweden). Blood was modeled as an incompressible fluid with a density of 1050 kg/m³ with a dynamic viscosity of 2.9 cP. The bundle was modeled as a single porous medium with the permeability quantified using a modified Blake-Kozeny equation.

TABLE 3
Summary of CFD studies

| | | | | Surface | Priming | vO₂ | vCO₂ |
|---|---|---|---|---|---|---|---|
| D | L | | Flow Rate | Area | Volume | [mL | [mL |
| [cm] | [cm] | D/L | [mL/min] | [cm₂] | [mL] | O₂/min] | CO₂/min] |
| 2.50 | 3.20 | 0.78 | 165.00 | 856 | 7.57 | 14.6 | 12.2 |
| 2.60 | 3.00 | 0.87 | 165.00 | 868 | 7.68 | 14.5 | 12.1 |
| 2.70 | 2.90 | 0.93 | 165.00 | 905 | 8.00 | 14.6 | 12.3 |
| 2.80 | 2.70 | 1.04 | 165.00 | 906 | 8.01 | 14.5 | 12.0 |
| 3.00 | 2.60 | 1.15 | 165.00 | 1002 | 8.86 | 14.8 | 12.6 |
| 3.10 | 2.40 | 1.29 | 165.00 | 988 | 8.73 | 14.6 | 12.3 |
| 3.20 | 2.30 | 1.39 | 165.00 | 1009 | 8.92 | 14.6 | 12.3 |
| 3.40 | 2.10 | 1.62 | 165.00 | 1040 | 9.19 | 14.6 | 12.2 |
| 3.60 | 2.00 | 1.80 | 165.00 | 1110 | 9.81 | 14.7 | 12.5 |
| 3.80 | 1.80 | 2.11 | 165.00 | 1113 | 9.84 | 14.5 | 12.2 |
| 4.00 | 1.70 | 2.35 | 165.00 | 1165 | 10.30 | 14.6 | 12.3 |

Resistance apparatus and measurement. The resistance apparatus included 3.5 cm inner diameter acrylic tube with height increments marked at 6, 10, 12, 14, and 16 cm above the outlet of the device. The bottom of the tube was sealed with an acrylic disk and a 3/16″ port was introduced tangent to the bottom of the column. A piece of connection tubing directed flow from the column to the device. The column and device were connected such that there was no vertical or horizontal gap between the outlet connector of the column and the inlet connector of the device. In a number of studied embodiments, the inlet connector of the device was a 3/16 inch commercially available barbed tubing port which had a through hole having a diameter of 3.556 mm. In other studied embodiments, a commercially available tubing port having a through hole with a 4.13 m diameter was used. A decrease in resistance of the device was achieved with the larger through hole. A 3 cm length of 3/16″ tubing was placed on the outlet connector of the device to control fluid flow via a tubing clamp (FIG. 2). The fluid was a fetal blood analogue made with carboxymethylcellulose sodium salt (Sigma Aldrich, St. Louis, MO) at a dynamic viscosity of 2.9±0.1 cP. The device was primed with the solution prior to column connection. After connection, the resistance fixture was filled with the blood analogue above the height mark of 16 cm. The tubing clamp was removed from the device outlet and the passage of fluid through the column and the device was video recorded. The time from an in-frame stopwatch (Traceable Stopwatch, Thomas Scientific, Swedesboro, NJ) was used to calculate the elapsed time for the blood analogue to pass from each height increment to the final height. The protocol was performed in triplicate.

In this experimental design the device will account for the majority of the resistance the fluid will encounter as it drains from the column. Flow resistance, R, integrated over the device in the vertical direction yields:

$$R = \frac{{P}_{0}}{Q} \qquad [ 25 ]$$

where Q is the volumetric flow rate, given by Q=V₀/A (superficial velocity, V₀, and column cross sectional area, A). The gage pressure, P₀, depends on the height of the fluid, h(t), in the column as a function of time:

P₀=pgh(t) [26]

where g is gravitational acceleration and p is the fluid density. The conservation of mass, V₀=−dh/dt, relates the two equations and can be integrated to yield:

$$ln ( \frac{{h}_{i}}{{h}_{f}} ) = \frac{ρ g}{A R} Δ t \qquad [ 27 ]$$

where Δt is the time span it takes the fluid to drop from an initial (hᵢ=16, 14, 12, or 10 cm) to final height (h_{f}=6 cm).

In Vitro Gas Exchange. Gas exchange was performed using abattoir adult bovine blood (8200811, Lampire Biological Laboratories of Pipersville, PA) heparinized upon collection (1000 IU/mL). The blood was treated with gentamicin (0.1 mg/mL) upon arrival in the laboratory and was used within 24 hours of the bleed date. A hemoglobin of 11±1 g/dL was achieved by diluting blood with phosphate buffered saline.

The test circuit (FIG. 3) included the device, two compliant blood reservoirs (MVR 800, Medtronic Minneapolis, MN), a Pedimag™ blood pump (Abbott Laboratories of Chicago, IL), and an Affinity oxygenator (Medtronic, Minneapolis, M). Both reservoirs were submersed in a heated water bath maintained at a temperature of 37±1 °C. The Pedimag recirculated blood through a single reservoir while the Affinity conditioned blood to EPI venous conditions (PCO₂=42.5±5 mmHg and PO₂=26.5±5 mmHg). Blood flow rate was maintained at 165 mL/min and was ultrasonically monitored using a Transonic flow probe (Transonic Systems Inc. of Ithaca, NY). Once conditioned, blood flow was directed to follow a single pass through the test circuit to the second empty reservoir. Three sweep gas flow rates, 750, 900, and 1000 mL/min, were randomly cycled through for a total of 3 samples per sweep gas flow rate. A WMA-4 CO₂ analyzer (PP Systems of Amesbury, MA) monitored the CO₂ concentration in the exhausted sweep gas from the device. Once gas exchange reached steady state, blood samples were taken from the device inlet and outlet for blood gas analysis (Rapidpoint 405 blood gas analyzer, Siemens of Deerfield, IL). Steady state was achieved when the concentration of CO₂ in the exhaust sweep gas changed by less than 10 ppm. Oxygenation and vCO₂ were calculated according to published equations and normalized to targeted venous conditions.

In Vitro Hemolysis. Hemolysis testing followed ATSM standard F1841-19. Blood was acquired and treated in the same manner as in-vitro gas exchange experiments. The control circuit included a Pedimag and a Paragon Neonatal (Chalice Medical of Nottinghamshire, UK) modified to exclude the heat exchanger. The experimental circuit include the oxygenator and a Pedimag. Pedimags were set to 1000±100 RPM resulting in both circuits having statistically equivalent flow rates (227±6 mL/min, p=0.1). Testing was performed for 6 hours with samples of hemoglobin (Rapidpoint 405 blood gas analyzer, Siemens, Deerfield IL), hematocrit (IEC Mb, Microcentrifuge, International Equipment CO.), protein concentration (Reichert TS 400 Refractometer, Reichert, Inc., Depew, NY), and plasma free hemoglobin (pfHb) evaluated hourly. To measure pfHb, whole blood was centrifuged (accuSpin Micro 17, Fisher Scientific, Hampton, NH) according to F1841-19 section 8.8 and spectrophotometrically analyzed (Genesys 10 UV-Vis Spectrophotometer, Thermo Fisher Scientific, Waltham, MA) at 540 nm. Absorbance values were correlated to pfHb concentration using a calibration curve generated from samples of known pfHb. A therapeutic index of hemolysis (TIH) and normalized index of hemolysis (NIH) were calculated according to standard formulas for both the control and experimental circuit.

The foregoing description and accompanying drawings set forth a number of representative embodiments at the present time. Various modifications, additions and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

1. A device for use in connection with a premature infant environment to achieve gas exchange, comprising:
a housing, a fiber bundle positioned within a fiber bundle compartment within the housing, the fiber bundle comprising a plurality of hollow gas permeable fibers, the plurality of hollow gas permeable fibers being adapted to permit diffusion of gas between blood and an interior of the plurality of hollow gas permeable fibers, the plurality of hollow gas permeable fibers being positioned such that blood flows around the plurality of hollow gas permeable fibers when flowing through the fiber bundle compartment, the device further comprising a gas inlet in fluid connection with the housing and in fluid connection with inlets of the plurality of hollow gas permeable fibers, a gas outlet in fluid connection with the housing and in fluid connection with outlets of the plurality of hollow gas permeable fibers, a blood outlet in fluid connection with the housing and in fluid connection with a first end of the fiber bundle and, a blood inlet in fluid connection with the housing and in fluid connection with a second end of the fiber bundle, the blood outlet being configured to be placed in fluid connection with the circulatory system of the premature infant, the blood inlet being configured to be placed in fluid connection with the circulatory system of the premature infant, wherein a maximum total resistance of the device is no greater than the resistance of a native placenta minus the resistance of a flow path connecting the device to the circulatory system of the premature infant.

2. The device of claim 1 wherein the maximum total resistance of the device is no greater than 100 (mmHg)(min)(L)⁻¹ minus the resistance of the flow path connecting the device to the circulatory system of the premature infant.

3. The device of claim 1 wherein the maximum total resistance of the device is no greater than 71 (mmHg)(min)(L)⁻¹.

4. The device of claim 3 wherein the fiber bundle has a surface area in the range of 560-1165 cm².

5. The device of claim 3 wherein the fiber bundle has a diameter-to-length ratio in the range of 0.78-3.64.

6. (canceled)

7. The device of claim 3 wherein a priming volume of the device is in the range of 5-30 ml.

8. The device of claim 3 wherein the device achieves a carbon dioxide removal rate of at least 8.5 mL/min and an oxygenation range of 8.5 mL/min at a flow rate of 165 mL/min.

9. The device of claim 1 wherein the plurality of hollow gas permeable fibers of the fiber bundle extend generally perpendicular to the direction of bulk flow of blood through the fiber bundle from the second end of the fiber bundle to the first end of the fiber bundle.

10. The device of claim 5 wherein the plurality of hollow gas permeable fibers is formed in at least one generally cylindrical bundle.

11. (canceled)

12. The device of claim 8 wherein bulk flow of blood through the fiber bundle is in a generally axial direction.

13. The device of claim 3 wherein a priming volume of the device is no greater than 30 ml.

14. The device of claim 3 wherein a normalized index of hemolysis (NIH) during operation is less than 0.05 g/100 L.

15. The device of claim 1 wherein the fiber bundle compartment comprises an inlet manifold at a first end thereof which is in fluid connection with the blood inlet and an outlet manifold at a second end thereof which is in fluid connection with the blood outlet.

16. The device of claim 15 wherein the wall of the inlet manifold is filleted and a channel of the inlet manifold extends generally tangentially from an axial end of the inlet manifold.

17. The device of claim 16 wherein the wall of the outlet manifold is filleted and a channel of the outlet manifold extends from the inlet manifold at a position below an axis of the fiber bundle compartment with respect to the gravity vector.

18. The device of claim 1 wherein the device is configured such that flow of blood through the device is driven by the heart of the premature infant.

19. A system comprising:
a device comprising a housing, a fiber bundle positioned within a fiber bundle compartment within the housing, the fiber bundle comprising a plurality of hollow gas permeable fibers, the plurality of hollow gas permeable fibers being adapted to permit diffusion of gas between blood and an interior of the plurality of hollow gas permeable fibers, the plurality of hollow gas permeable fibers being positioned such that blood flows around the plurality of hollow gas permeable fibers when flowing through the fiber bundle compartment, the device further comprising a gas inlet in fluid connection with the housing and in fluid connection with inlets of the plurality of hollow gas permeable fibers, a gas outlet in fluid connection with the housing and in fluid connection with outlets of the plurality of hollow gas permeable fibers, a blood outlet in fluid connection with the housing and in fluid connection with a first end of the fiber bundle and, a blood inlet in fluid connection with the housing and in fluid connection with a second end of the fiber bundle, the blood outlet being configured to be placed in fluid connection with the circulatory system of a premature infant, the blood inlet being configured to be placed in fluid connection with the circulatory system of the premature infant
a flow path to connect the device to the circulatory system of the premature infant, and
a source of sweep gas,
wherein a maximum total resistance of the device is no greater than the resistance of a native placenta minus the resistance of the flow path connecting the device to the circulatory system of the premature infant.

20. The system of claim 19 wherein the flow path includes a cannulation system for cannulation of at least one of the umbilical arteries of the premature infant and a cannulation system for cannulation of the umbilical vein of the premature infant.

21. A method of providing care to a premature infant, comprising:
placing a device in fluid connection with the circulatory system of the premature infant, the device comprising a housing, a fiber bundle positioned within a fiber bundle compartment within the housing, the fiber bundle comprising a plurality of hollow gas permeable fibers, the plurality of hollow gas permeable fibers being adapted to permit diffusion of gas between blood and an interior of the plurality of hollow gas permeable fibers, the plurality of hollow gas permeable fibers being positioned such that blood flows around the plurality of hollow gas permeable fibers when flowing through the fiber bundle compartment, the device further comprising a gas inlet in fluid connection with the housing and in fluid connection with inlets of the plurality of hollow gas permeable fibers, a gas outlet in fluid connection with the housing and in fluid connection with outlets of the plurality of hollow gas permeable fibers, a blood outlet in fluid connection with the housing and in fluid connection with a first end of the fiber bundle and, a blood inlet in fluid connection with the housing and in fluid connection with a second end of the fiber bundle, the blood outlet being configured to be placed in fluid connection with the circulatory system of the premature infant, the blood inlet being configured to be placed in fluid connection with the circulatory system of the premature infant via a flow path in fluid connection with the blood inlet and the blood outlet of the device, wherein a maximum total resistance of the device is no greater than the resistance of a native placenta minus the resistance of a flow path connecting the device to the circulatory system of the premature infant.

22. The method of claim 21 wherein the flow path includes a cannulation system for cannulation of at least one of the umbilical arteries of the premature infant and a cannulation system for cannulation of the umbilical vein of the premature infant.
